(19) United States
(12) Patent Application Publication
(10) Pub. No.: US 2016/0296629 A1
(43) Pub. Date: Oct. 13, 2016

(54) IRON COMPLEXING AGENT AND USES THEREOF IN THE TREATMENT AND PREVENTION OF COLORECTAL CANCER

(71) Applicant: THE UNIVERSITY OF BIRMINGHAM, Birmingham (GB)

(72) Inventors: Chris Tselepis, Birmingham (GB); John S. Fossey, Birmingham (GB); Rama Byravan, Birmingham (GB)

(73) Assignee: THE UNIVERSITY OF BIRMINGHAM, Birmingham (GB)

(21) Appl. No.: 15/037,779

(22) PCT Filed: Nov. 28, 2014
(86) PCT No.: PCT/GB2014/053529

§ 371 Date: May 19, 2016

(30) Foreign Application Priority Data
Nov. 28, 2013 (GB) 1320992.9

(51) Int. Cl. A61K 47/48; A61K 31/192; A61K 9/00; A61K 31/4196

(52) U.S. Cl. CPC A61K 47/4823; A61K 31/4196; A61K 31/192; A61K 9/0053

(57) ABSTRACT

A complex comprising a non-absorbable portion attached to an iron chelator moiety, a composition comprising the complex and the use of the complex in the treatment of colorectal cancer. In one embodiment the non-absorbable portion is a polymer such as a polysaccharide, including chitosan, chitin, cellulose or pectin. In one embodiment the iron chelator moiety comprises at least one functional group selected from catechol, hydroxamate or carboxylate, or any combination thereof.

The present invention relates to a complex comprising a non-absorbable portion. In particular, the invention relates to a complex comprising a non-absorbable portion attached to an iron chelator, and uses thereof.

Colorectal cancer is one of the major causes of cancer death in Western Societies. In the UK, the disease is the second commonest cause of cancer mortality. Approximately 37,000 cases are diagnosed each year, and around 16,000 deaths per year are caused by the disease.

A large number of factors are linked to an increased risk of developing colorectal cancer. These include age, a genetic predisposition, a family history of colorectal cancer, a personal history of inflammatory bowel disease, and lifestyle-related factors. Lifestyle-related factors such as obesity, smoking, high alcohol intake, diet and a lack of exercise have all been linked to colorectal cancer.

In particular, the high prevalence of this type of cancer in Western cultures has been linked to the Western diet which is high in processed and red meat. It is not exactly understood which components present in these types of foods contribute to the increased cancer risk. It has previously been suggested that the cooking and processing of meats at high temperatures creates carcinogenic chemicals. The high levels of fat in meat have also been hypothesised to contribute to the development of cancer.

Another hypothesis is that high levels of iron (Fe), such as those found in red meat, may be linked to colorectal cancer, although previous in vitro studies and animal models investigating the effects of iron on colorectal cancer have produced mixed results, and it has remained unclear how iron mediates carcinogenesis.

Given the prevalence of the disease in today's society, it is clear that furthering our understanding of the causes of colorectal cancer and finding new ways to prevent, manage or even cure the disease is of extreme importance.

The present invention has been devised with these issues in mind.

According to a first aspect of the present invention, there is provided a complex comprising a non-absorbable portion attached to an iron chelator moiety.

By “non-absorbable” it will be understood that the portion is non-absorbable and non-digestible in the gastrointestinal (GI) tract. In other words, the portion cannot be digested, fermented or broken down in the Gl tract, nor can it be absorbed from the Gl tract into the blood stream.

A non-absorbable portion is advantageous where the complex of the invention is intended to be used therapeutically, particularly in the treatment or prophylaxis of colorectal cancer. Research by the inventors suggests that colorectal cancer is associated with an excess of unabsorbed colonic luminal iron, rather than an excess of systemic iron. It is therefore believed that colorectal cancer may be treated or prevented by depleting the luminal pool of iron through the use of an iron chelator. However, if the iron chelator is absorbed through the colon and into the blood stream it could exacerbate the risk of developing cancer and or anaemia. By attaching the iron chelator to a non-absorbable portion this problem is circumvented by preventing absorption of the iron chelator into the bloodstream.

In some embodiments, the complex consists of a non-absorbable portion attached to one or more iron chelator moieties. In some embodiments, a non-absorbable portion is attached to multiple iron chelator moieties. It will be understood that where multiple iron chelator moieties are attached to the non-absorbable portion, the iron chelator moieties may all be the same, or they may be different.

In some embodiments, the non-absorbable portion is suitable for administration to humans and/or other animals. In further embodiments, the non-absorbable portion is biocompatible. By “biocompatible” it will be understood that the non-absorbable portion does not have a toxic effect on humans and/or other animals.

In some embodiments, the non-absorbable portion is a polymer. The polymer may be a linear polymer or a branched polymer. In further embodiments, the non-absorbable portion is a dendritic polymer (also referred to herein as a ‘dendrimer’). Examples of suitable dendrimers include poly(amino amine) (PAMAM), hyperbranched polymers, poly(ethylene glycol) (PEG) core dendrimers and polypropylenimine dendrimers. However, it will be appreciated that any non-absorbable dendrimer which can be functionalized with an iron chelator may be used.

In some embodiments, the non-absorbable portion is a polysaccharide. The polysaccharide may be alginate, chitosan, chitin (a polymer of /V-acetylglucosamine), cellulose or pectin.

In some embodiments, a single iron chelator moiety is attached to a single non-absorbable portion. However, it will be appreciated that a single non-absorbable portion may be attached to multiple iron chelator moieties, particularly when the non-absorbable portion is a polymer. For example, in embodiments wherein the non-absorbable portion is a polysaccharide, there may be at least 1 iron chelator moiety for every 3, every 5 or every 10 sugar monomers.

It will be understood that a chelator is a molecule which binds free metal ions and removes them from solution. Metal ions bound by a chelator molecule are effectively inactivated because they are no longer available to react with other chemical species. An iron chelator is a chelator that binds Fe ions. “Fe ions”, as used herein, will be understood as referring to both ferric and ferrous ions, unless otherwise stated.

In some embodiments the iron chelator moiety is iron specific. By “iron specific”, it will be understood that the iron chelator moiety preferentially binds (i.e. is selective for) Fe ions (Fe(II) and/or (Fe(III)) in the presence of other metal ions. In some embodiments, the iron chelator moiety selectively binds Fe(III) ions in the presence of other ions. A chelator moiety which is selective for iron is particularly beneficial since a number of metal ions will be present in the gut and non-specific binding of these to the chelator may compromise the ability of the chelator moiety to reduce luminal iron levels.

As will be known by those skilled in the art, the strength of the interaction between a chelator and an ion may be indicated by the stability constant, which is an equilibrium constant for the formation of a chelator-ion complex. In some embodiments, the stability constant of the iron chelator moiety with Fe(III) is at least 1×10²⁵, at least 1×10³⁰ or at least 1×10³⁵. In some embodiments, the stability constant of the iron chelator moiety with Fe(II) is at least 1×10¹⁰, at least 1×10¹², at least 1×10¹⁵ or at least 1×10¹⁸.

In some embodiments, the iron chelator moiety is capable of binding Fe(II) and/or Fe(III) ions in the presence of calcium. Thus, the affinity of the iron chelator moiety for Fe(II) and/or Fe(II) ions may be higher that the affinity of the chelator for Ca(II) ions. This property is particularly advantageous for chelation of iron in the colon (luminal iron). While it may be desirable to reduce the level of luminal iron, it may be undesirable to deplete the calcium which is also present in the colon.

Furthermore, by attaching the iron chelator moiety to the non-absorbable portion, the secondary and tertiary structure of the non-absorbable portion may be altered. This may ablate any calcium-binding capability of a non-absorbable portion such as alginate.

In some embodiments, the stability constant of the iron chelator moiety with Ca(II) is less than 1×10²⁰, less than 1×10¹⁸, less than 1×10¹⁶, less than 1×10¹² or less than 1×10¹⁰.

The iron chelator moiety may be derived from a naturally occurring iron chelator. In some embodiments, the iron chelator moiety is a siderophore, or a derivative thereof. A siderophore is an iron-chelating compound produced by microorganisms in order to scavenge iron from the environment. Siderophores can be classified according to their functional groups (sometimes referred to as ‘ligands’) which form a complex with the iron. The siderophore may be a catecholate (e.g. enterobactin), a hydroxamate (e.g. desferrioxamine) or a carboxylate. Siderophores comprising a mixture of ligands are also known. Enterobactin advantageously has a particularly high affinity for Fe(III) (K=10⁵²). Desferrioxamine also has a high affinity for Fe(III) over other trivalent species.

Non-siderophore catechol-containing molecules may also function as the iron chelator moiety. Thus, in some embodiments, the iron chelator moiety comprises at least one, at least two or at least three catechol groups. Examples of suitable catechol-containing compounds include dopamine, norepinephrine and catechin, but it will be appreciated that any catechol-containing compound may be incorporated into the complex. In further embodiments, the iron chelator moiety is (or is derived from) hydrocaffic acid.

In some embodiments, the iron chelator moiety comprises at least one, at least two or at least three hydroxamic acid (hydroxamate) groups (i.e. RCONR′OH). In some embodiments, the iron chelator moiety comprises at least one, at least two or at least three carboxylic acid (carboxylate) groups (i.e. RCOOH). In further embodiments, the iron chelator moiety comprises at least one, at least two or at least three functional groups selected from catechol, hydroxamate or carboxylate, or any combination thereof.

The iron chelator moiety may be a synthetic molecule. In some embodiments, the iron chelator moiety is an aminocarboxylate, such as EDTA or DTPA, or a derivative thereof. However, in some embodiments, the iron chelator moiety is not EDTA or DPTA. In some embodiments, the iron chelator moiety comprises deferiprone (3-hydroxy-1,2-dimethylpyridin-4(1H)-one) or a derivative thereof.

In some embodiments, the iron chelator moiety comprises the structure of formula I

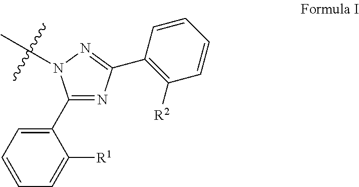

wherein at least one of R¹ and R² is a hydroxyl (OH) group. In some embodiments, both R¹ and R² are hydroxyl groups. It will be appreciated that the structure of formula I may be modified to include one or more additional functional groups such as alkyl, acyl, amine, amide, alcohol, aryl, ether, acid and/or sulphur groups. Additional functional groups may be included to modify the chelation properties of the molecule, or its physical properties such as solubility.

In some embodiments, the iron chelator moiety is derived from Deferasirox and comprises the structure shown in formula II:

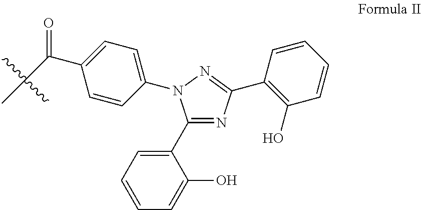 [SMILES: *C(=O)c1ccc(-n2nc(-c3ccccc3O)nc2-c2ccccc2O)cc1]

Conveniently, Deferasirox has great selectivity for Fe(III) over divalent ions. This means that it is able to chelate luminal iron without depleting levels of other important ions such as Ca²⁺ and Zn²⁺. Deferasirox is also particularly advantageous for therapeutic use due to the absence of severe side effects.

The iron chelator moiety may be attached to the non-absorbable portion by a covalent bond. The iron chelator moiety may be directly bonded to the non-absorbable portion. For example, a covalent bond may be formed between a functional group (such as an acid, acid chloride, amide, alcohol, ether, nitrogen or sulphur group) of the iron chelator and a functional group of the non-absorbable portion. Alternatively, the complex may additionally comprise a linker between the non-absorbable portion and the iron chelator moiety.

The linker may be any molecule which is suitable for reliably attaching the iron chelator moiety to the non-absorbable portion. The linker may comprise from 2 to 20, from 5-15 or from 5 to 10 atoms. In some embodiments, the linker comprises or consists of a linear chain of at least 4, at least 6 or at least 8 atoms. Such chains are useful for providing flexibility. The linker may comprise a hydrocarbon chain, optionally including one or more polar groups such as O, OH or NH₂. Such groups help to enhance the solubility of the complex. In some embodiments, the linker comprises one or more ethylene glycol groups.

The complexes of the invention may be used to chelate iron in both medical and non-medical environments.

According to a second aspect of the invention, there is provided a composition comprising the complex of the first aspect of the invention.

In some embodiments, the composition is adapted for selective delivery of the complex to the colon.

By “selective delivery of the complex to the colon”, it will be understood that the iron chelator moiety of the complex is unable to bind iron until it reaches the colon. This prevents the complex from chelating iron in the small intestine, if the composition is ingested.

Colonic delivery of the complex may be achieved by oral or rectal administration of the composition.

For rectal administration, the composition may be in the form of a suppository incorporating the complex and a carrier such as cocoa butter, or in the form of an enema.

For oral administration, the composition may be in the form of discrete units such as capsules, cachets, tablets or lozenges, each containing a predetermined amount of the complex; in the form of a powder or granules; in the form of an emulsion, suspension in an aqueous liquid or non-aqueous liquid. The composition may also be in the form of a bolus, electuary or paste.

For oral administration, colonic delivery of the complex may be achieved by ingestion of the composition wherein the iron chelator is in an inactivated form, or wherein the complex or iron chelator moiety is in a form which otherwise prevents the iron chelator moiety from binding iron until it reaches the colon. Alternatively, the iron chelator moiety may be ingested in the form of a precursor or prodrug, which is activated or modified in vivo such that the active iron chelator moiety is only released in the colon.

In some embodiments, the composition is encapsulated by a coating which remains intact while the composition passes through the stomach and small intestine, but which degrades in the colon. In a series of embodiments, colonic delivery is achieved using a coating which is pH sensitive, time-dependent, pressure-dependent or degradable by intestinal bacteria. In some particular embodiments, the composition is encapsulated by a biodegradable coating. The coating may be a polymer. In other embodiments, the coating involves colon-targeted microsponges, or a microbially-triggered osmotic pump. The composition may be targeted to the colon by a combination of the above strategies.

In some embodiments, the coating is a biodegradable polysaccharide. Such polysaccharides will be known to those skilled in the art, and may include albizia gum, alginates, amylose, arabinogalactan, cellulose, chondroitin sulphate, curdlan, cyclodextrin, dextran,furcelleran, galactomannan, gellan gum, guar gum, hyaluronic acid, inulin, kara gum, karaya gum, locust bean gum, scleroglucan, starch, pectin, pulluvan or xylan.

The composition of the present invention may further comprise one or more additional pharmaceutically acceptable ingredients such as excipients, bulking agents, diluents, buffers, flavouring agents, binders, surface active agents, thickeners, lubricants and preservatives.

According to a third aspect of the invention, there is provided the complex of the first aspect of the invention, or a composition of the second aspect of the invention, for use in the treatment or prophylaxis of colorectal cancer.

It will be understood that “colorectal cancer” refers to cancer of the colon, rectum or appendix.

The treatment or prophylaxis may comprise administering a therapeutically effective amount of the complex or composition to a subject in need thereof. The subject may have been diagnosed as having colorectal cancer, or the subject may have been identified as having a high risk of developing colorectal cancer. Some conditions, such as inflammatory bowel disease (IBD), increase the risk of a subject developing colorectal cancer. The treatment or prophylaxis may therefore comprise administering the complex or composition to a subject having IBD. The subject may be human or animal.

“Inflammatory bowel disease” (IBD) refers to a group of inflammatory conditions of the colon and small intestine and includes Crohn's disease, ulcerative colitis, collagenous colitis, lymphocytic colitis, ischaemic colitis, diversion colitis, Behcet's disease and indeterminate colitis.

In some embodiments, the complex is administered in an amount of from 1 mg to 2 g, from 50 mg to 1 g, from 100 mg to 500 mg or from 150 mg to 350 mg per day. It will be appreciated that where the complex is administered as part of a composition, the amount of composition administered to the subject will be sufficient to deliver the appropriate dose of the complex.

The complex (or the composition comprising the complex) may be administered as a single dose or as multiple doses (e.g. 2, 3 or 4 doses at intervals of e.g. 3, 6 or 8 hours). The complex (or the composition comprising the complex) may be administered orally or rectally.

Embodiments of the invention will now be described by way of example and with reference to the accompanying Figures, in which:

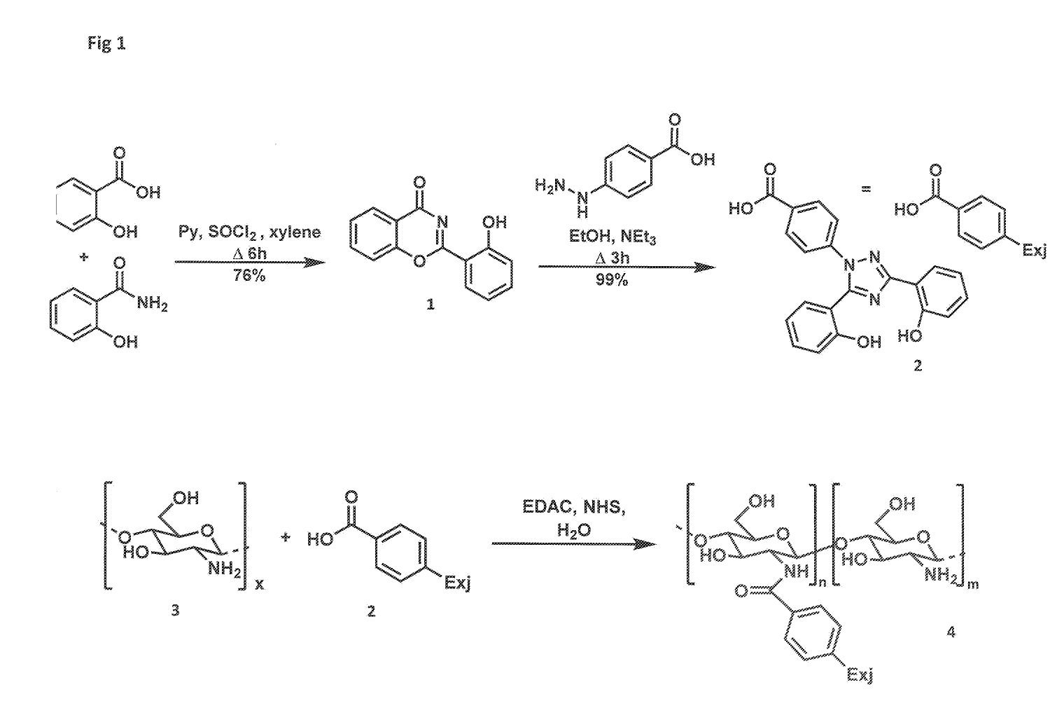

FIG. 1 is a reaction scheme showing the preparation of a deferasirox-chitosan complex;

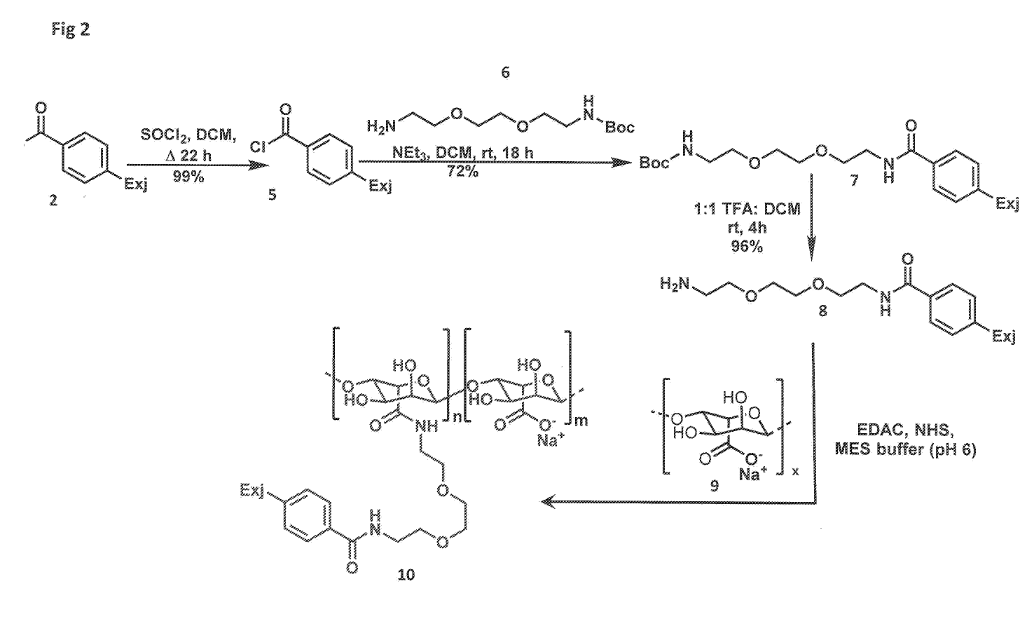

FIG. 2 is a reaction scheme showing the preparation of a deferasirox-alginate complex comprising a linker;

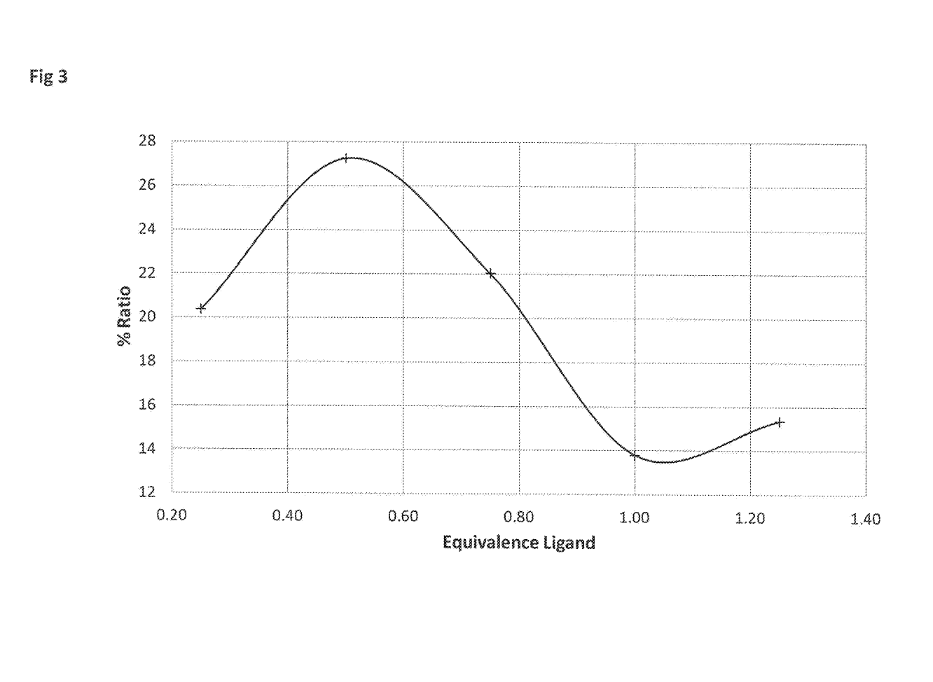

FIG. 3 is a plot of the molar modification of alginate with varying chelator concentration;

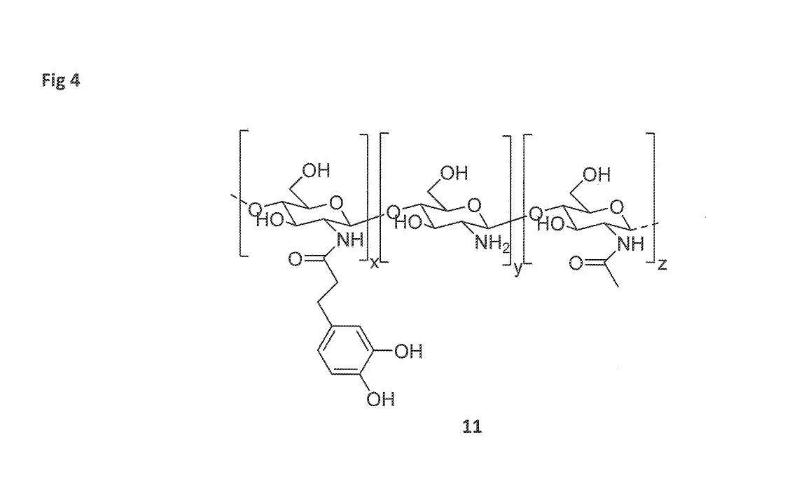

FIG. 4 is shows the chitosan-hydrocaffeic acid complex (11)

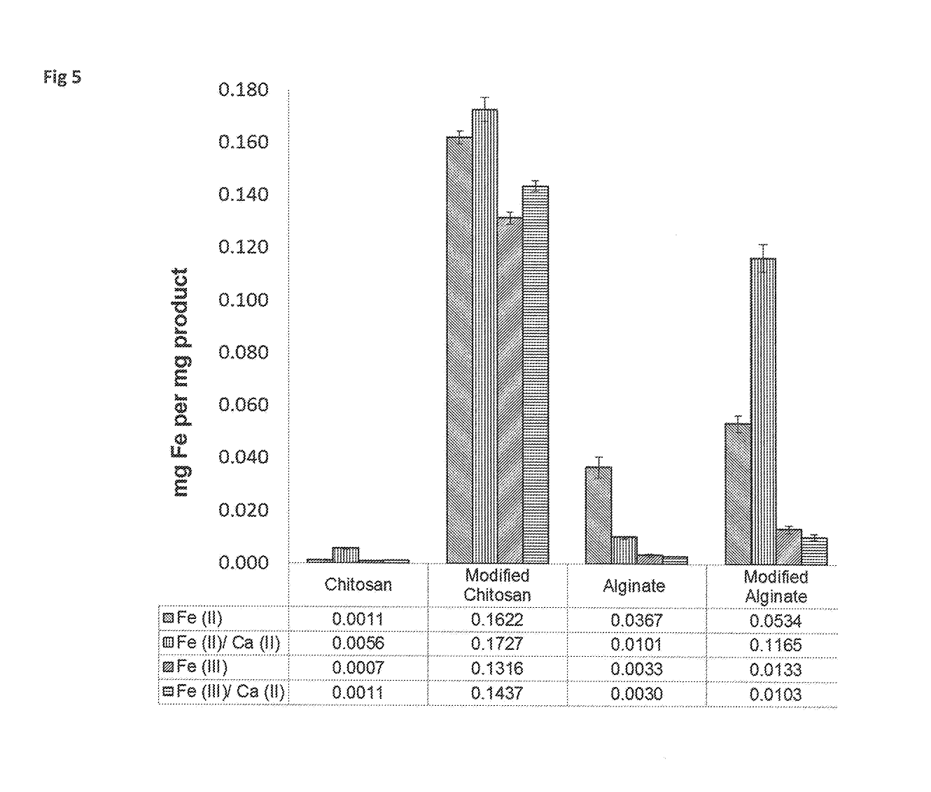

FIG. 5 is a graph showing the Fe binding to modified and unmodified polymers, as revealed by Ferrozine assay;

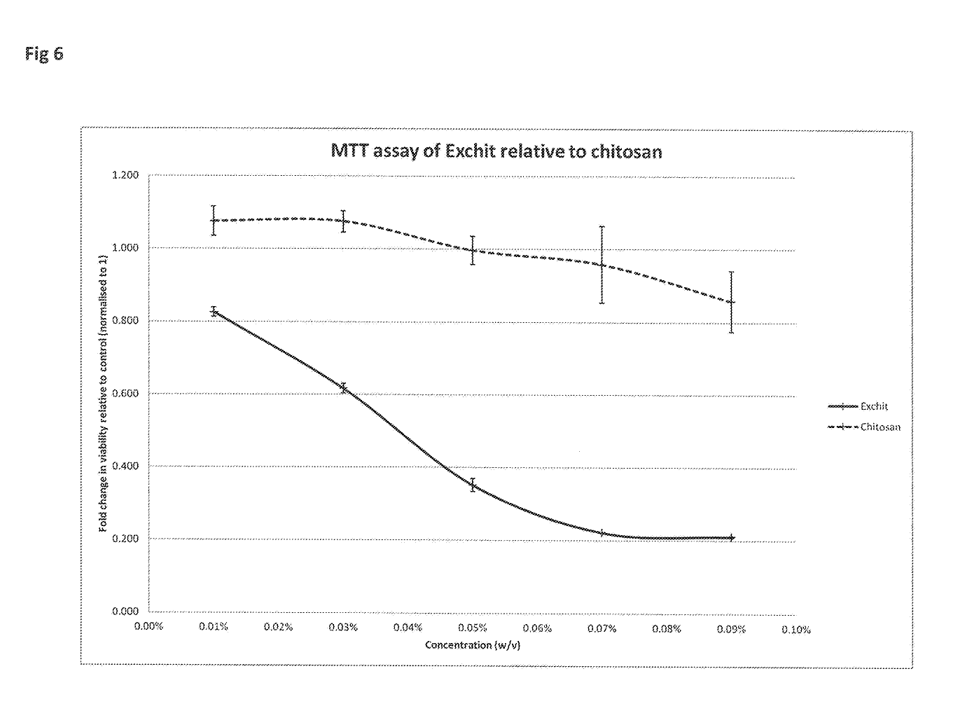

FIG. 6 shows the results of an MTT assay comparing the deferasirox-chitosan complex (Exchit) and chitosan;

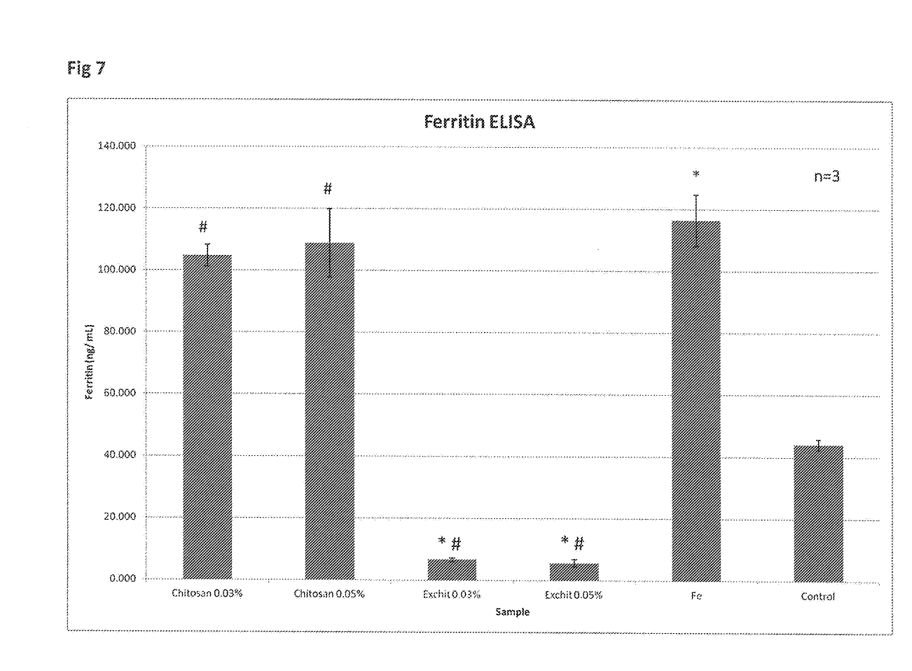

FIG. 7 is a Ferritin ELISA for the deferasirox-chitosan complex (Exchit) and chitosan;

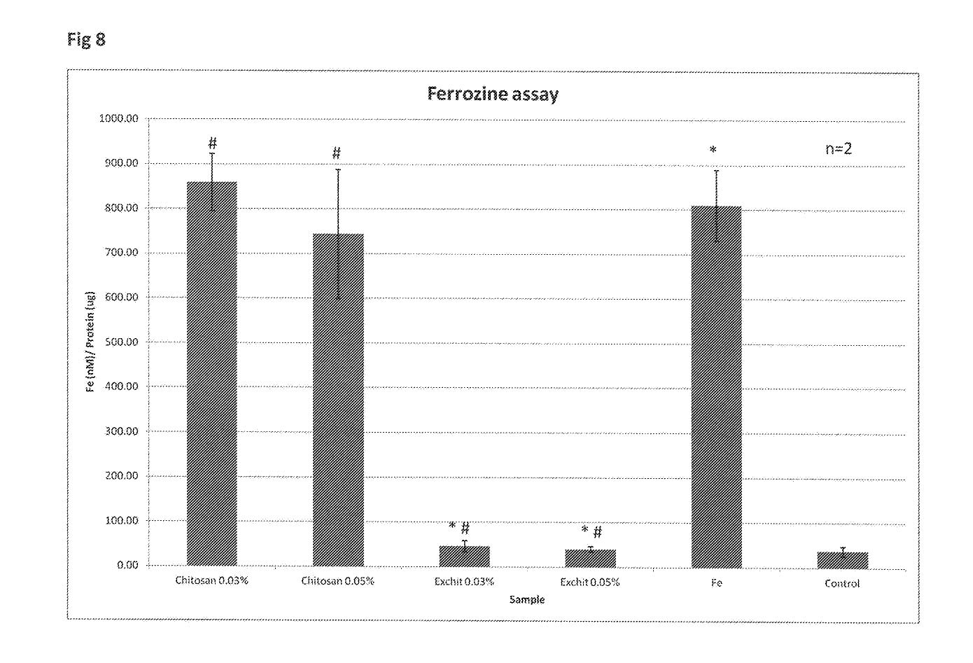

FIG. 8 is a Ferrozine assay for the deferasirox-chitosan complex (Exchit) and chitosan;

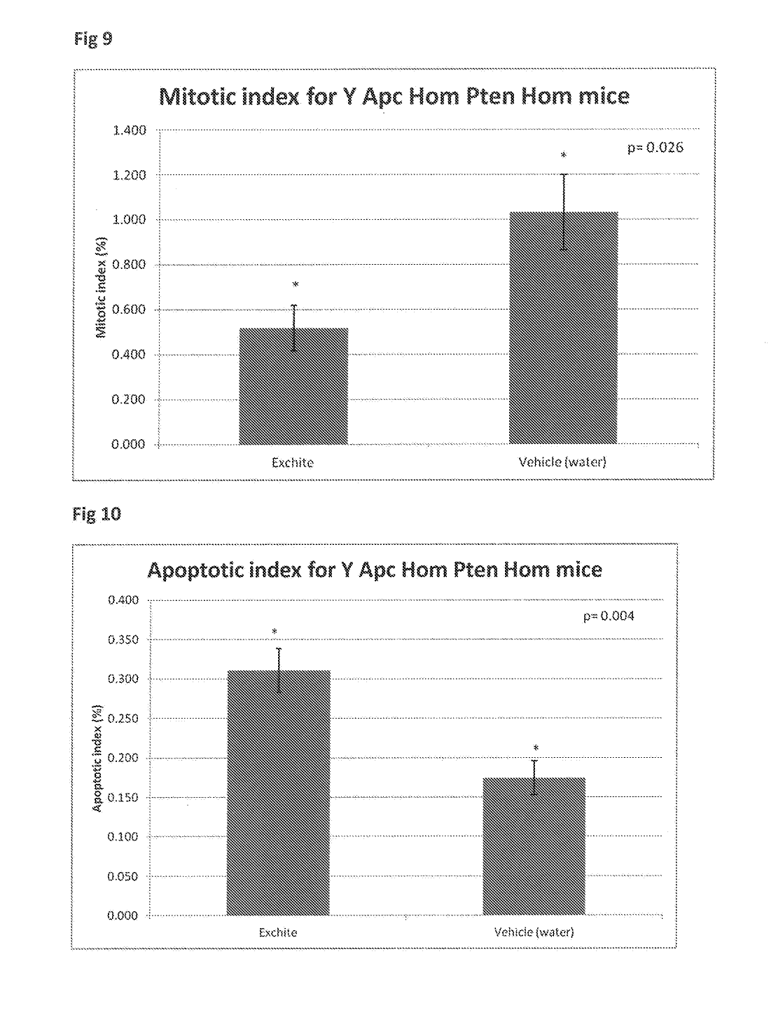

FIGS. 9 and 10 are charts showing mitotic and apoptotic index respectively for Y Ape Horn Pten Horn mice given the deferasirox-chitosan complex (Exchit).

### EXAMPLE 1

### Chitosan-Deferasirox Complex

A chitosan-deferasirox complex was prepared according to the reaction scheme shown in FIG. 1.

1.1 Preparation of Deferasirox

Salicylic acid (6.04 g, 43.75 mmol), salicylamide (5.00 g, 36.46 mmol) and pyridine (0.37 mL, 4.63 mmol) were heated at reflux in xylene (18.00 mL) for 15 min. Thionyl chloride (5.83 mL, 80.21 mmol) was added with vigorous stirring over a period of 4 h, with further stirring for 16 h at room temperature. Xylene was removed by concentration in vacuo, and resulting solid residue was suspended in ethanol (15.00 mL) and acetic acid (0.36 mL). The mixture was heated to reflux and cooled to room temperature. The solid precipitate was isolated and dried to yield 2-(2-Hydroxyphenyl)-benzo-4H-[1,3]-oxazin-4-one (1) (6.59 g, 27.54 mmol, 76%) as yellow solid.

4-hydrazino-benzoic acid (1.40 g, 9.19 mmol), and triethylamine (1.28 mL, 9.19 mmol) were added to ethanol (40.00 mL) and refluxed for 15 min until all components have dissolved. To the clear solution was added (1) (1.40 g, 9.19 mmol) which was further refluxed for 2 h. After cooling to room temperature, water (5.00 mL) was added until perturbation was observed. The mixture was concentrated to 50% of total volume under reduced pressure, and aqueous 6 M HCl (31.00 mL) was added. The resulting precipitate was isolated and dried to yield 4-[3,5-Bis(2-hydroxyphenyl)-1,2,4-triazol-1-yl]benzoic acid (deferasirox) (2) (3.12 g, 8.36 mmol, 99%) as dark yellow solid.

1.2 Preparation of the Complex

EDAC. HCl (0.616 g, 3.214 mmol) and NHS (0.370 g, 3.314 mmol) in deionised water (150.00 mL) was added to deferasirox (2) (1.00 g, 2.678 mmol) dissolved in a few drops of DMSO. Chitosan (3) was then added (0.434 g, 2.678 mmol of monomer units) and solution stirred at r.t. for 18.5 h. Reaction solution was transferred to semi-permeable cellulose membrane and dialysed in deionised water for five days, with the water changed regularly. Water was lyophilised to yield the chitosan-deferasirox complex (4) as pale brown film (0.109 g).

### EXAMPLE 2

### Alginate-Deferasirox Complex

An alginate-deferasirox complex comprising a glycol linker was prepared according to the reaction scheme shown in FIG. 2.

2.1 Preparation of Linker

Boc anhydride (1.000 g, 1.053 mL, 4.582 mmol) in DCM (5.00 mL) was added portion wise over 2 h at room temperature to 2,2(ethylenedioxy)bis(ethylamine) (4.074 g, 4.01 mL, 27.489 mmol) in DCM (10.00 mL). This was further stirred at room temperature for 5 h and solvent was removed. Water (30.00 mL) was added to the residue and extracted with DCM (6×20.00 mL), the combined organic extracts were dried (MgSO₄) and concentrated in vacuo to yield the linker Tert-butyl (2-(2-(2-aminoethoxy)ethoxy)ethyl)carbamate (6) as pale yellow oil (0.982 g, 3.957 mmol, 86%).

2.2 Addition of Linker to the Iron Chelator

Thionyl chloride (0.20 mL, 2.687 mmol) was added to deferasirox (2Error! Reference source not found.) (0.100 g, 0.268 mmol) in DCM (20.00 mL), and solution refluxed for 22 h. The residue was concentrated in vacuo to yield 4-(3,5-bis(2-hydroxyphenyl)-1H-1,2,4-triazol-1-yl)benzoyl chloride (5) as dark brown solid (0.104 g, 0.268 mmol, 99%)

(5) (0.090 g, 0.230 mmol) was added to (6) (0.077 g, 0.310 mmol) in DCM (20.00 mL), followed by triethylamine (0.032 mL, 0.230 mmol). The reaction mixture was stirred for 18 h at room temperature, quenched by the addition of saturated NaHCO₃ solution (20.00 mL) and extracted with DCM (3×20.00 mL). The combined organic extracts were dried (MgSO₄), concentrated and purified by silica plug (5% MeOH, 1% NEt₃ in CHCl₃) to yield (7) as pale yellow solid (0.100 g, 0.166 mmol, 72%).

TFA (2.00 mL) was added to (7) (0.052 g, 0.086 mmol) in DCM (2.00 mL) and stirred at room temperature for 4 h. Saturated NaHCO₃ solution (20.00 mL) was added and subsequently extracted with DCM (3×10 mL), dried (MgSO₄) and concentrated in vacuo to yield N-(2-(2-(2-aminoethoxy)ethoxy)ethyl)-4-(3,5-bis(2-hydroxyphenyl)-1H-1,2,4-triazol-1-yl)benzamide (8) as yellow solid (0.041 g, 0.081 mmol, 96%).

2.3 Preparation of Alginate-Linker-Chelator Complex

A solution of sodium alginate LFR5/60 (9) (0.250 g, 1.262 mmol), EDAC.HCl (0.242 g, 1.262 mmol), and NHS (0.145 g, 1.262 mmol) in MES buffer (30.00 mL, 0.10 M, pH 6) was prepared. To this was added (8) (0.635 g, 1.262 mmol) dissolved in MES buffer (10.00 mL, 0.10 M, pH 6) and the reaction mixture was stirred at room temperature for 62 h. The precipitate was isolated by centrifuge and dried to yield the alginate-deferasirox complex (10) as pale brown powder (0.785 g).

2.4 Effect of Ligand Concentration on % Modification of Alginate

The effect of ligand concentration on % modification of alginate was determined using Phenol/sulphuric acid assay. Phenol solution (0.05 mL, 80 % w/w) was added to sugar solution (2.00 mL), and then concentrated sulphuric acid was added rapidly (5.00 mL) ensuring that the stream of acid was being directed against the liquid surface rather than against the side of vial for good mixing. The solution was left to stand for 10 min, and then shaken and placed for 20 min in a water bath at 30° C. Readings were taken by UV-vis spectroscopy, measured at 490 nm for hexoses and 480 nm for pentoses and uronic acids. The amount of sugar was determined by reference to a standard curve constructed for the particular sugar under examination. Blanks were prepared by substituting distilled water for the sugar solution.

The results of the assay (FIG. 3) revealed that % modification was highest when 0.50 equivalence of ligand (linker-chelator conjugate) was used.

### EXAMPLE 3

### Chitosan-Hydrocaffeic Acid Complex

A solution of hydrocaffeic acid (0.500 g, 2.745 mmol), EDAC.HCl (0.631 g, 3.294 mmol) and NHS (0.379 g, 3.294 mmol) was prepared in water: ethanol mixture (1:1 15.00 mL:15.00 mL). To this was added a solution of chitosan (0.445 g, 2.745 mmol) in acidic aqueous media (60.00 mL water acidified by HCl to pH 5), and the resulting reaction mixture was stirred at room temperature for 16 h. Reaction solution was transferred to semi-permeable cellulose membrane and dialysed in acidic aqueous media (pH 5) for three days, with the aqueous media changed regularly. The dialysed solution was freeze dried to yield the chitosan-hydrocaffeic acid complex (11) (FIG. 4) as a brown solid.

### EXAMPLE 4

### Fe Binding Experiments

The binding of Fe to modified (i.e. complexed) and non-modified polymers in the presence and absence of Ca(II) was investigated using Ferrozine assay and Atomic Absorption Spectroscopy.

Methodology

Dialysis Experiments

Polymer solutions (chitosan and alginate at 0.1% w/v, modified polymers at lower concentrations due to poor solubility in acidic media, 10 mL) were sealed into a dialysis membrane and incubated in ferrous iron (FeSO₄.7H₂O) (0.20 mM, 750.00 mL), ferrous iron (FeSO₄.7H₂O) (0.20 mM, 750.00 mL) with calcium (CaCl₂.2H₂O) (2.00 mM, 750.00 mL), ferric iron (FeCl₃.6H₂O) (0.20 mM, 750.00 mL), and ferric iron (FeCl₃.6H₂O)(0.20 mM, 750.00 mL) with calcium (CaCl₂.2H₂O) (2.00 mM, 750.00 mL) for 2 h. Dialysis in acidic supernatant required addition of HCl (12 M, 0.75 mL in 750.00 mL). When washing step was conducted, the sealed dialysis membrane was washed in deionised water (750.00 mL) for 2 h. All experiments were performed in triplicate unless otherwise stated.

Ferrozine Assay

To assess for iron content, a Ferrozine assay was utilised whereby a Ferrozine stock solution consisting of sodium ascorbate (20 mL, [0.23 M]), Ferrozine (18 mL, [10 mM]) and sodium acetate (84 mL, [2 M]) was made in deionised water. Each polymer sample (200 μL) was mixed with Ferrozine stock (600 μL) and HCl (12 M, 50 μL), ensuring that reference samples are also prepared in the same way and absorbance read on a plate reader at λ=550 nm.

Atomic Absorption Spectroscopy

Dialysed samples were either: concentrated and redissolved in HNO₃ (16 M, 0.50 mL) and HCl (3 M, 9.50 mL) or HNO₃ (16 M, 1.00 mL) was added directly to the samples. Samples were heated to 80° C and sonicated for 2-4 h, and iron content analysed by flame atomic absorption spectrometer.

NB: Increased viscosity of alginate samples caused the readings to fluctuate rapidly. The fluctuations were dampened by dilution of the samples at 1 in 5 dilution factor with water. Calibration standards were also diluted accordingly.

Results

The results of the Ferrozine assay are shown in FIG. 5. The modified chitosan (i.e. chitosan-deferasirox complex) was found to have significantly improved binding of both Fe(II) and Fe(III) compared to chitosan alone. The presence of Ca(ll) did not appear to affect Fe(II) or Fe(III) binding, indicative of the chelator being iron-specific.

An improvement of Fe(II) binding by modified alginate (i.e. alginate-linker-deferasirox complex) was observed compared to alginate alone. Fe(II) binding was found to be greater than Fe(III) due to the formation of Fe(III) oxides which precipitate out of solution.

Similar results were observed using AAS.

### Example 5

### In Vitro Cellular Studies

The colorectal cell line RKO was cultured in the presence of 0.03% w/v and 0.05% w/v modified chitosan. It was observed that the presence of the modified chitosan elicited suppression of cellular viability. Cellular iron levels were also significantly suppressed compared to cells cultured with chitosan alone. The modified chitosan was also able to significantly suppress any iron mediated induction in ferritin protein expression (ferritin being a surrogate marker of cellular iron) compared to iron alone and iron+chitosan. Not only was the high affinity of the chitosan-chelator complex for iron surprising, but the ability of the complex to strip iron from cells was particularly unexpected. This suggests that the complex is able to interact with the cell surface and remove iron from the cell via an as yet uncharacterized mechanism. This could have significant implications for the therapeutic uses of the complexes of the invention.

### EXAMPLE 6

### Synthesis of a Library of Iron Chelators

Modifications to the chelator moiety were made by utilization of COOH chemistry or by changing the hydrazine moiety in order to build the library of chelators shown in Tables 1-3.

TABLE 1
Reaction scheme

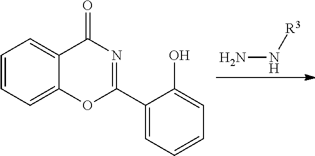

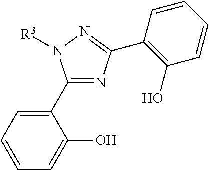

| Iron chelator | Conditions | R³ | Yield |
|---|---|---|---|
| Deferasirox (2) | EtOH, Δ, 2 h | PhCOOH | 99% |
| A | MeOH, Δ, 2.5 h | NO₂ | 50% |
| B | MeOH, Δ, 2 h | C₂H₄OH | 43% |

TABLE 2
Reaction scheme

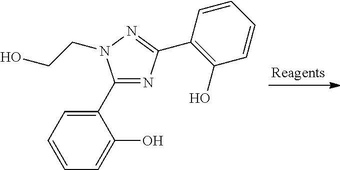 [SMILES: OCCn1nc(-c2ccccc2O)nc1-c1ccccc1O]

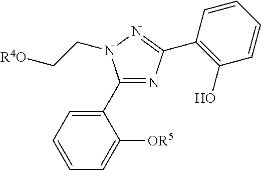

| Iron | | | | | |
|---|---|---|---|---|---|
| chelator | Reagents | Conditions | R⁴ | R⁵ | Yield |
| C | 1.0 PhCOOH, 1.2 | DCM, rt, 18 h | COPh | H | 18% |
| | DCC, 0.3 DMAP | | | | |
| D | 1.2 TsCl, 2.0 NEt₃ | DCM, rt, 70 h | Ts | H | 20% |
| E | 1.2 TsCl, 2.0 NEt₃ | DCM, rt, 70 h | Ts | Ts | 12% |

TABLE 3
Reaction scheme

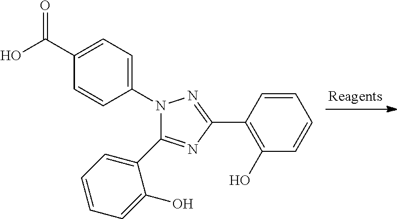 [SMILES: O=C(O)c1ccc(-n2nc(-c3ccccc3O)nc2-c2ccccc2O)cc1]

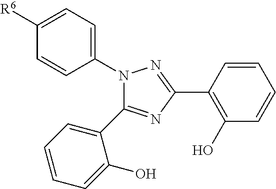

| Iron chelator | Reagents | Conditions | R⁶ | Yield |
|---|---|---|---|---|
| F | MeOH, H₂SO₄ | Δ, 18 h | COOMe | 99% |
| G | EtOH, H₂SO₄ | Δ, 23 h | COOEt | 99% |
| H | 1.2 NHS, 1.2 DCC, 2.0 | DCM, Δ, 3 h | CONHⁱPr | 83% |
| | ⁱPrNH₂ | | | |

### EXAMPLE 7

### Cellular Iron Binding

Iron binding of the modified chitosan polymer 4 (Exchite) was tested by in vitro cellular assays. Cell studies were conducted with RKO cells which is a poorly differentiated colon carcinoma cell line. Cell lines were cultured in an incubator at 37° C and 5% CO2 atmosphere. Cells were grown in Dulbecco's Modified Eagles Medium (DMEM) supplemented with 10% (v/v) Foetal Calf Serum (FCS), 1% (v/v) Penicillin/Streptomycin.

MTT assay was used to determine the toxicity of compound on cells and to determine the appropriate concentration to use in further cellular studies.

10 MTT assay: RKO cells (1×105 cells/mL) were seeded into 6-well plates (Corning) at 2.00 mL per well. After cells were allowed to adhere for 24 h, they were treated with the appropriate concentration of Exchit/chitosan in cell culture media at 2.00 mL per well and further incubated for 24 h. Experiments were done in triplicate for each concentration and control was conducted in cell culture media alone. MTT solution (100 μL of 0.005 g MTT/1.00 mL PBS solution) was added to each well and incubated for 3 h. Cell culture media was removed and DMSO (1.00 mL) was added to each well to dissolve formazan crystals. Each well was plated out in triplicate into 96-well plate at 100 μL/well and was read on a Victor Spectrophotometer at 490 nm. The fold change in cellular viability was calculated relative to control, normalised to one.

MTT assay was conducted at 0.01%. 0.03%, 0.05%, 0.07%, 0.09% w/v, and a difference in cellular viability was observed as viability decreased with increasing concentrations, as shown in FIG. 6. Chitosan has no effect on cellular viability at low concentrations and may decrease viability by 10% at higher concentrations. There is a clear trend with Exchite: at 0.01% the cellular viability is 0.8 which lowers and levels off at 0.2 at 0.07%.

Once the appropriate compound concentration was determined by MTT assay, iron binding studies were conducted by incubating RKO cells with iron in the presence of the compound to determine whether the iron chelator will inhibit iron from entering cells. Intracellular iron was measured by western blot and ferritin ELISA to quantitatively measure ferritin levels, which is the intracellular iron storage protein. If there is an uptake of iron by cells, the ferritin levels will increase. Total intracellular iron was measured by the ferrozine assay.

24 h stimulations for Western blots and ELISA: RKO cells (1×10⁵ cells/mL) were seeded into 6-well plates (Corning) at 2.00 mL per well. Stimulation solutions were made up with 100 μM FeSO₄.7H₂O and 10 μM sodium ascorbate, in 0.03% and 0.05% w/v Exchite/Chitosan in cell culture media. Positive control of 100 μM FeSO₄.7H₂O and 10 μM sodium ascorbate only and negative control of media only was used. After cells were allowed to adhere for 24 h, they were treated with the stimulation solutions (2.00 mL per well) and further incubated for 24 h. Experiments were done in triplicate for each concentration. The media was removed and cells washed with PBS buffer (1.00 mL per well×3), and lysed with 100 μL RIPA buffer. Western blot and ELISA was conducted according to standard protocols.

Western blot was performed with lysates as prepared above, β-Actin is used as an internal marker. Ferritin expression is observed for chitosan 0.03% and 0.05% relative to Fe only control. However, ferritin expression is greatly diminished for Exchit 0.03% relative to Fe only control and normal control. Greater ferritin expression observed with Fe positive control and both concentrations of Chitosan suggesting that Chitosan has no Fe binding effect. There is significantly less ferritin produced in the presence of Exchite suggesting that this is binding iron and preventing it from entering cells.

The results of the Ferritin ELISA (FIG. 7) show a similar trend to the Western blot. The Exchite suppresses iron uptake significantly whereas chitosan alone does not seem to have any effect on the iron uptake of cells.

The results of the Ferrozine assay (FIG. 8) show very similar trends as observed with ferritin expression from Western blots and ELISA. Chitosan does not seem to affect iron uptake however there is greatly diminished iron uptake of cells in the presence of Exchite, down to the levels of the negative control.

Ferrozine assay: RKO cells (1×105 cells/mL) were seeded into 6-well plates (Corning) at 2.00 mL per well. Stimulation solutions were made up with 100 μM FeSO4.7H2O and 500 μM sodium ascorbate, in 0.03% and 0.05% w/v Exchite/Chitosan in cell culture media. Positive control of 100 μM FeSO4.7H2O and 500 μM sodium ascorbate only and negative control of media only was used. After cells were allowed to adhere for 24 h, Cells were serum starved (1 h) and treated with the stimulation solutions (2.00 mL per well) and further incubated for 1 h. Experiments were done in triplicate for each concentration. The media was removed and cells washed with PBS buffer (1.00 mL per well×3), and lysed with 150 μL HEPES buffer. Ferrozine assay was conducted according to standard protocols.

### EXAMPLE 8

### Mouse Experiments

Initial mice experiments were done with wild type mice to check for toxicity. They were given daily 200 uL gavages of Exchite for five days at varying concentrations: 0.01%, 0.10%, 0.25%, 0.50% and 1% w/v. After five days, none of the mice showed any adverse reactions so further mice experiments were done with 1% w/v Exchite.

Ape hom Pten hom mice were used as a model of intestinal cancer where the mice have an induced Ape and Pten deletion. A dose of 1% w/v Exchite (200 uL) was given to them on Monday/Wednesday/Friday until they became sick. Whilst there was no apparent survival advantage of mice given Exchite there were cellular alterations in terms of cellular apoptosis and mitosis (FIGS. 9 and 10).

The murine intestines were stained with phosphohistone (mitosis), caspase (apoptosis), and H&E (cellular morphology, mitosis and apoptosis). The slides were scored by counting 25 crypts for positive staining per mouse, with 6 mice taken per group. The total number of positively stained cells is expressed as a percentage of total cells counted as an average of 25 crypts in 6mice per group, and is given as an apoptotic or mitotic index.

FIG. 8 show a reduced mitotic index (cellular multiplication) from both phosphohistone stain and H&E for the Exchite compared to vehicle, which is statistically significant. Also, the caspase stain shows that there is an increase of apoptosis (programmed cell death) for Exchite compared to the vehicle which is also statistically significant (FIG. 10). Therefore, treatment with Exchite is reducing cellular mitosis and stimulating cell death with the net effect likely to be less tumourigenesis.

1. A complex comprising a non-absorbable portion attached to an iron chelator moiety.

2. The complex of claim 1, consisting of a nonabsorbable portion attached to one or more iron chelator moieties.

3. The complex of claim 1, wherein the non-absorbabie portion is biocompatible.

4. The complex of claim 1, wherein the non-absorbable portion is a polymer.

5. The complex of claim 4, wherein the non-absorbable portion is a polysaccharide.

6. The complex of claim 5, wherein the polysaccharide is alginate, chitosan, chitin, cellulose or pectin.

7. The complex of claim 1, wherein the iron chelator moiety is iron specific.

8. The complex of claim 1, wherein the iron chelator moiety has a higher affinity for Fe(II) and/or Fe(III) ions than for Ca(II) ions.

9. The complex of claim 1, wherein the iron chelator moiety comprises at least one functional group selected front catechol, hydroxamate or carboxylate, or any combination thereof.

10. The complex of claim 1, whetem the iron chelator moiety comprises the structure of formula I, 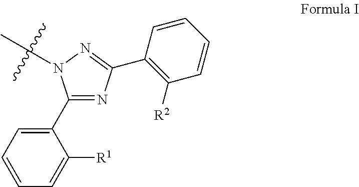
wherein at least one of R¹ and R² is a hydroxyl group.

11. The complex of claim 10, wherein the iron chelator moiety comprises the structure of formula II:

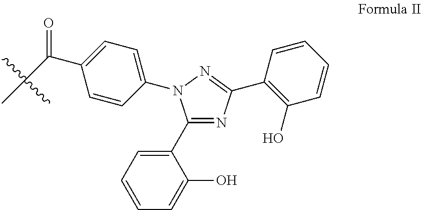 [SMILES: *C(=O)c1ccc(-n2nc(-c3ccccc3O)nc2-c2ccccc2O)cc1]

12. The complex of claim 1, wherein the complex further comprises a linker between the non-absorbable portion and the iron chelator moiety.

13. A composition comprising the complex of claim 1.

14. The composition of claim 13, adapted for selective delivery of the complex to the colon.

15. The composition of claim 14, wherein the composition is encapsulated.

16. (canceled)

17. A method of treatment or prophylaxis of colorectal cancer, the method comprising administering a therapeutically effective anmrnt of the complex of claim 1 to a subject in need thereof.

18. The method of claim 17, wherein the complex is administered orally.

19. The method of claim 17, wherein the subject has been identified as having a high risk of developing colorectal cancer, or wherein the subject has inflammatory bowel disease (IBD).

20. A method of treatment or prophylaxis of colorectal cancer, the method comprising administering a therapeutically effective amonnt of the composition of claim 13 to a subject in need thereof.

21. The method of claim 20, wherein the subject has been identified as having a high risk of developing colorectal cancer, or wherein the subject has inflammatory bowel disease (IBD).
